(19) United States
(12) Patent Application Publication
(10) Pub. No.: US 2020/0115659 A1
(43) Pub. Date: Apr. 16, 2020

(54) POLYORGANOSILOXANE-COATED AND/OR AMORPHOUS SILICON DIOXIDE-COATED TEXTILE LAUNDRY ARTICLE

(71) Applicant: Henkel AG & Co. KGaA, Dusseldorf (DE)

(72) Inventors: Sascha Schaefer, Mettmann (DE); Svenja Neuhaus, Duisburg (DE)

(21) Appl. No.: 16/599,132

(22) Filed: Oct. 11, 2019

(30) Foreign Application Priority Data
Oct. 12, 2018 (DE) 10 2018 125 333.8

(51) Int. Cl. C11D 3/37; C11D 3/00; C11D 3/12; C11D 17/04; C11D 3/22; C11D 11/00; A61K 9/70; A61K 8/02

(52) U.S. Cl. CPC C11D 3/373; C11D 3/0036; C11D 3/124; C11D 17/046; A61K 8/0208; C11D 3/3715; C11D 11/0017; A61K 9/7007; C11D 3/222

(57) ABSTRACT

A coated textile laundry article may include a water-insoluble textile substrate that is exposed to at least one modified or unmodified polyorganosiloxane and/or at least one amorphous silicon dioxide. Furthermore, the coated textile laundry article may be used for washing laundry and/or to prevent graying of laundry. A method for machine or manual washing of laundry using the coated textile laundry article and a method for its preparation is also described.

### CROSS-REFERENCE TO RELATED APPLICATION

The present application claims priority to German Patent Application Serial No.: 10 2018 125 333.8 according to 35 U.S.C. § 119, which was filed on Oct. 12, 2018; which is incorporated herein by reference in its entirety and for all purposes.

### TECHNICAL FIELD

The present invention relates to washing laundry and/or preventing graying of laundry using a coated textile laundry article comprising a water-insoluble textile substrate, and more specifically relates to a textile substrate that is exposed to at least one modified or unmodified polyorganosiloxane and/or at least one amorphous silicon dioxide.

### BACKGROUND

With regard to more sustainable and environmentally friendly washing processes, it is important to obtain acceptable cleaning with as few ingredients as possible. Many of the formulations used today for detergents contain, in addition to substances that allow the removal of dirt and the holding of the dirt in the wash liquor, also substances to reduce graying, a process that results in clouding when washing, in particular, light textiles and thus in a reduction in perceived cleanliness of the laundry. Holding the dirt in the wash liquor and preventing it from graying is an important performance feature of modern washing systems.

Such washing systems are now available to consumers in a variety of forms of presentation. In addition to powders and granules, this presentation also includes liquids, gels or sachets (tablets or filled bags). In particular, portion packs fulfilled the consumer's desire for simplified dosage. Detergent tablets or bags filled with liquid and/or solid detergents, however, also have a number of disadvantages. In particular, tableted presentation forms are characterized by their high compression often by a delayed disintegration and thus a delayed release of their ingredients. Therefore, the dirt dispersed in the wash liquor has more time to segregate on a woven fabric.

In the case of sachets filled with liquid detergents, there is the disadvantage that incompatible ingredients cannot be formulated with each other. Often, the shell materials used (for example, polyvinyl alcohol) do not dissolve in time and remain as residues on the washed laundry. Likewise, the dissolved polymer components reach the environment with the wash liquor, which should be avoided.

### SUMMARY

It has been surprisingly found that polyorganosiloxanes or amorphous silicon dioxides are suitable to be compatible with a textile substrate and to prevent or reduce the graying of laundry. Also, the textile laundry article may avoid an increased entry of polymers in the wash liquid; the textile laundry article may be disposed of as a substrate/cloth in the household waste after the washing process has been completed. “Wash liquid” as used herein may include an aqueous phase impregnated with the detergent, which is commonly referred to as ‘suds’, since the wash liquid may be worked into a froth or foam.

In a first non-limiting aspect, a coated textile laundry article may include a water-insoluble textile substrate that is exposed to at least one modified or unmodified polyorganosiloxane and/or at least one amorphous silicon dioxide.

Furthermore, in a second non-limiting aspect, the use of the coated textile laundry article may be used for washing laundry and/or for preventing graying of laundry.

In a third non-limiting aspect, a method for machine or manual washing of laundry may include using a textile laundry article.

Finally, in a fourth non-limiting aspect, a method for producing a coated textile laundry article may include:
providing a water-insoluble textile substrate,
subjecting the water-insoluble textile substrate to at least one modified or unmodified polyorganosiloxane and/or at least one amorphous silicon dioxide and optionally a detergent composition that can flow when heated above 30° C. and optionally a further composition on at least one zone of the water-insoluble textile substrate; and
cooling and hardening the modified or unmodified polyorganosiloxane and/or the amorphous silicon dioxide and the optional detergent composition and the optional further composition on the water-insoluble textile substrate.

### DETAILED DESCRIPTION

“At least one,” as used herein, refers to 1 or more, for example 2, 3, 4, 5, 6, 7, 8, 9 or more. In the context of the non-limiting embodiments described herein, this indication does not refer to the absolute amount or number of a molecule or ingredient, but to the nature of the ingredient. “At least one polyorganosiloxane” therefore means, for example, that at least one type of polyorganosiloxane is present, but it may also contain two or more different types of polyorganosiloxanes. At least one does not refer to the amount of polyorganosiloxane molecules present in the composition.

Numbers given herein without decimals refer to the full value, one decimal place, each. For example, “99%” stands for “99.0%.”

The term “about” or “approximately” in the context of a numerical value refers to a variance of ±10% based on the given numerical value, preferably ±5%, more preferably ±1%.

Unless indicated otherwise, all percentages are indicated in terms of weight % (wt. %). Numerical ranges that are given in the format “from x to y” include the cited values “x” and “y.” If several numerical ranges are indicated in this format, it is self-evident that all ranges that result from the combination of the various endpoints are also included.

The above described and further aspects, embodiments, features, and advantages will become apparent to a person skilled in the art through studying the detailed description and claims below. In this case, each feature of an embodiment can be used in any other embodiment. Furthermore, it will readily be understood that the examples contained herein are intended to describe and illustrate but not to limit the non-limiting embodiments and that, in particular, the non-limiting embodiments are not limited to these examples.

The facts, objects and embodiments described below, which are described for the coated textile laundry article, are also applicable to the manufacturing method and methods for machine or manual washing of laundry and use.

The coated textile laundry article comprises a water-insoluble textile substrate where the textile substrate is exposed to at least one modified or unmodified polyorganosiloxane.

In various non-limiting embodiments, the modified or unmodified polyorganosiloxane may be or include, but is not limited to polyalkylsiloxanes, fluorosilicones, ether-modified silicones, polyether-modified silicones, and combinations thereof.

Non-limiting unmodified polyalkylsiloxanes may have a structure according to formula I:

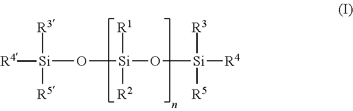

R¹ and R² independent of one another being a C1-C10 alkyl radical, such as ethyl, methyl or propyl;
R³ to R⁵ and R^{3′} to R^{5′} independent of one another being a C1-10 alkyl radical or a halogen atom, such as —F; such as ethyl, methyl or propyl; and
n being an integer from 10 to 1800, such as from 20 to 1600, or alternatively from 150 to 1500, or alternatively from 300 to 1350.

Alternative non-limiting unmodified polyalkylsiloxanes are cyclic polyalkylsiloxanes having a structure of the formula:

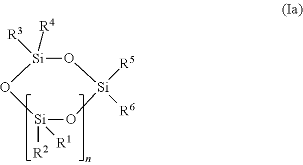

R¹ to R⁶ independent of one another being a C1-C10 alkyl radical, such as ethyl, methyl or propyl; and
n being an integer from 1 to 1000, such as from 2 to 100, or alternatively from 2 to 50, or alternatively from 2 or 3. In a non-limiting embodiment, the unmodified polyalkylsiloxane is dodecamethylcyclohexasiloxane.

Non-limiting fluorosilicones may have a structure of formula II:

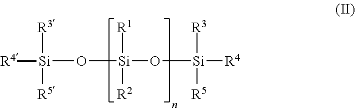

R¹ and R² independent of one another being a C1-C10 alkyl radical, such as ethyl, methyl, propyl, or —(CH₂)ₘCFₚH₃₋ₚ, wherein m=1 to 10, p=1-3;
R³ to R⁵ and R^{3′} to R^{5′} independent of one another being a C1-10 alkyl radical or a halogen atom, such as —F; such as ethyl, methyl, or propyl; and
n being an integer from 10 to 1800, such as from 20 to 1600, or alternatively from 150 to 1500, or alternatively from 300 to 1350, where
in at least one unit n, such as from 2 to 1500, or alternatively from 5 to 1000 units, a —(CH₂)ₘCFₚH₃₋ₚ radical is contained.

Non-limiting polyalkylsiloxanes modified with ether or polyether may have a structure according to formula III:

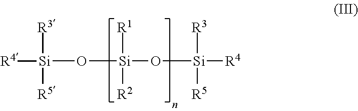

R¹ and R² independent of one another being a C1-C10-alkyl radical, such as ethyl, methyl, propyl, or —(CH₂)ₘO(CₚH₂ₚO)_{q}H, where m=1 to 10, p=1-5, such as 2 or 3, q=1 to 1500, or alternatively from 3 to 150;
R³ to R⁵ and R^{3′} to R^{5′} independent of one another being a C1-10 alkyl radical or a halogen atom, such as —F; such as ethyl, methyl, or propyl; and
n being an integer from 10 to 1800, such as from 20 to 1600, alternatively from 150 to 1500, or alternatively from 300 to 1350, where at least one unit n, such as from 2 to 1500, or alternatively 5 to 1000 units, a —(CH₂)ₘO(CₚH₂ₚO)_{q}H radical is contained.

Suitable polyorganosiloxanes may be commercially available from Dow Corning, for example under the trade name AC-8066.

In various non-limiting embodiments, at least one amorphous silicon dioxide is included.

In general, all amorphous silicon dioxides known to the person skilled in the art are suitable. “Amorphous silicon dioxide” is noncrystalline or low crystalline silicon dioxide. Amorphous silicon dioxide has no long-range order and thus differs from crystalline silicon dioxide such as quartz. Amorphous silicon dioxide includes quartz glass made of amorphous silicon dioxide. This is made by melting crystalline silicon dioxide (naturally occurring quartz) into a non-crystalline form. Alternatively, synthetic quartz glass can be made by pyrolysis of silicon tetrachloride or vaporized quartz to form minute droplets of amorphous quartz glass which fuse into an articulated particle structure.

Such a form of synthetic quartz glass is also known by the term “fumed silica.” Amorphous silicon dioxide can also be precipitated from the solution to form small porous particles that can fuse together in chains. Such a form of silica is known as silica gel, which can be used to form silica aerogels. Adjusting the pH of the solution can keep the particles separate to form larger individual particles, commonly referred to as precipitated silicon dioxide or silicon dioxide sols, which are all forms of amorphous silicon dioxide. In various non-limiting embodiments, the amorphous silicon dioxide is solid at 22° C. and 1 bar. In various non-limiting embodiments, the amorphous silicic acid may be or include, but is not limited to, silicic acid aerogels, silica gel, fumed silicic acid, and combinations thereof.

In various non-limiting embodiments, especially when it is fumed silicic acid, the amorphous silicic acid may be treated with a surface treatment. Such surface treatments include, but are not limited to, polydimethylsiloxane coatings and vinylalkoxysilanes. In general, the substances used for surface treatment do not constitute more than 10% by weight (“wt %”) of the total treated amorphous silicon dioxide and may generally be less than 5% by weight. In a non-limiting embodiment, at least one treated amorphous silicon dioxide, such as at least one treated fumed silicic acid, is used.

In a non-limiting embodiment, there is at least one modified or unmodified polyorganosiloxane and at least one amorphous silicon dioxide. At least one cyclic polyalkylsiloxane of the formula (Ia) as defined above and/or at least one treated amorphous silicon dioxide may be present. In a non-limiting embodiment, the modified or unmodified polyorganosiloxane may be contained relative to the amorphous silicon dioxide in a weight ratio of 0.1:99 to 10:90, such as 0.8:99.2 to 1.2:98.2.

The washing article may be permanently equipped with the modified or unmodified polyorganosiloxane and/or amorphous silicon dioxide.

“Permanent finish” is understood to be a permanent chemical and/or physical change of the water-insoluble substrate which imparts to it certain properties of use, in the present case graying-inhibiting properties. This change can be made to the fibers, which later form the water-insoluble textile substrate, or to the water-insoluble textile substrate.

The finishing of the textile substrate with graying-preventing properties is carried out by applying at least one modified or unmodified polyorganosiloxane and/or at least one amorphous silica to the textile substrate. This is fiber-reactive in one non-limiting embodiment and, with the fibers of the water-insoluble textile substrate, forms an ionic or covalent bond, such as a van-der-Waals bond. The finishing of the fibers can be done, for example, by means of a so-called “direct” method, in which the fibers may be treated with an application liquid, that is, for example, with a solution, dispersion or the like of the polyorganosiloxane and/or amorphous silicon dioxide. During treatment, the polyorganosiloxane(s) and/or amorphous silicon dioxide(s) used from the application liquid uniformly draws on the treated fibers. Alternatively, the water-insoluble textile substrate in the form of a non-woven or a woven fabric can be dipped into the application liquid or said textile substrate can be printed or sprayed with the application liquid. The polyorganosiloxane and/or amorphous silicon dioxide may be applied before, after or together with the optional detergent composition and/or further composition.

In further non-limiting embodiments, the modified or unmodified polyorganosiloxane may have a molecular weight from 150 to 50,000 g/mol or from 1,000 to 35,000 g/mol or from 2,500 to 25,000 g/mol. The molecular weight may be determined by gel permeation chromatography using polystyrene standards in a non-limiting embodiment.

In non-limiting embodiments, the modified or unmodified polyorganosiloxane may be contained in 0.001 to 50% by weight or 1.0 to 10.0% by weight, based on the total weight of the coated textile laundry article. In other non-limiting embodiments, the total weight of modified or unmodified polyorganosiloxanes is from 0.001 to 50% by weight, or 1.0 to 10.0% by weight, based on the total weight of the coated textile laundry article.

In non-limiting embodiments, the amorphous silica is contained in 0.001 to 50% by weight or 1.0 to 10.0% by weight, based on the total weight of the coated textile laundry article. In non-limiting embodiments, the total weight of amorphous silicon dioxide is from 0.001 to 50% by weight, or 1.0 to 10.0% by weight, based on the total weight of the coated textile laundry article.

The textile substrate may be a woven or non-woven fabric. Fabrics and non-woven fabrics have high durability and good resistance, so that these textile substrates can withstand the high mechanical loads during the washing process, especially in a washing machine. In non-limiting embodiments, the textile substrate is composed of cellulose (for example cotton), polyester, polyethylene, polypropylene, polyethylene terephthalate, polybutylene terephthalate, rayon, polyacrylic, polylactic acid, lyocell, viscose or nylon or mixtures thereof. In a non-limiting embodiment, the textile substrate contains at least two different materials, one material being unmodified polypropylene. Polypropylene fibers can bind grease and thus additionally counteract graying.

In a further non-limiting embodiment, at least one further composition is solidified on the substrate. The substrate may include a detergent composition in a non-limiting embodiment. In addition to the detergent composition, another, for example, a textile and/or skin care composition may be present on the textile substrate. The laundry article can thus be extended by a further functionality. The compositions may be solidified in a non-limiting embodiment.

In a non-limiting example, the detergent composition may have a color that is different from the color of the further composition in order to visually show that the coated textile laundry article has a plurality of functionalities.

The detergent composition and the further composition may be arranged side by side on the textile substrate. In another non-limiting embodiment, the detergent composition and the further composition are arranged at least partially overlapping on the textile substrate.

The fibers or the textile substrate may additionally be reacted with a coupling component, for example epichlorohydrin, and then with a basic nitrogen compound. Subsequent quaternization gives a dirt-trapping finish to the fibers.

In a further non-limiting embodiment, the fibers or the textile substrate are additionally provided with anionic groups, for example carboxyl groups and/or sulfonate groups, and subsequently reacted with the dirt-trapping compounds.

The dirt-trapping compound may have a cationic charge. In non-limiting embodiments, the dirt-trapping compound may be a cationic polymer. Non-ionic dirt-trapping compounds, for example, can be converted into cationically charged compounds with the aid of cationic coupling groups.

The soil-collecting compound may be or include, but is not limited to polyfunctional quaternary ammonium compounds, such as polyquaternium-6, polyquaternium-10 and cationic polygalactomannan derivatives; acrylic polymers, such as superabsorbent polyacrylates; the polyesters and/or the heterocyclic polymers, such as polyvinylpyrrolidone, polyamine N-oxide polymers, polyvinylimidazole; and copolymers of vinylpyrrolidone, vinylimidazole, vinyloxazolidone, vinylpyridine, and vinylpyridine N-oxide.

For trapping different types of dirt, the textile substrate may be equipped with two or more dirt-trapping compounds.

The dimensions of the fabric substrate should allow for easy handling of the laundry article, for example, 10 cm×10 cm or 20 cm×20 cm, although other sizes are possible. The textile substrate and thus the laundry article may be of any surface shape, accordingly, for example, square, rectangular, triangular, round, oval, heart-shaped, letter or word-shaped or the reproduction of a brand. The textile substrate may be of any color or substantially white; its surface may be substantially smooth or textured due to manufacturing. The textile substrate and thus the laundry article may have a surface area (relative to one side) of 40 cm² to 800 cm². In a non-limiting embodiment, the textile substrate is roughened at least on parts of its surfaces and thus has a surface enlargement, where a better and more stable absorbing of the polyorganosiloxanes may be achieved. Due to the increase in surface area, a larger amount of the polyorganosiloxanes can be applied or added, so that there is a larger number of binding sites for molecules which have a graying action to be fixed on or to the textile substrate. This increases the performance of the coated textile laundry article with respect to its graying-inhibiting properties.

In another non-limiting embodiment, the textile substrate has antimicrobial finish in addition to the grayness inhibiting finish. The antimicrobial finish can be applied to or on the textile substrate, for example, by incorporating silver threads during manufacture of the textile substrate or by applying silver compounds, such as for example, SILVERPLUS® (ex Rudolf Chemie).

A solidified detergent composition may be included on the textile substrate.

A detergent composition to be applied to the textile substrate, such as when it is to be applied as a melt, contains, at room temperature, solid or wax-like constituents, e.g. surfactants and organic polymers being expected, which can be melted at elevated temperature. In addition, the detergent composition for washing agents or laundry aftertreatment agents may contain conventional ingredients.

A detergent composition to be applied to the textile substrate may include builders, surfactants, bleaches, bleach catalysts, bleach activators, enzymes, sequestering agents, electrolytes, pH regulators, polymers with special effects, such as soil release polymers, dye transfer inhibitors, anti-crease agents and shape-retaining agents, and other adjuvants, such as optical brighteners, fungicides, antioxidants, UV absorber foam regulators, bleach activators, dyes, antimicrobial agents, and fragrances.

A non-limiting detergent composition may include one or more surfactants, such as anionic surfactants, nonionic surfactants and mixtures thereof, but also cationic surfactants. Suitable non-ionic surfactants may include alkyl glycosides, ethoxylation, and/or propoxylation products of alkyl glycosides or linear or branched alcohols each having from 12 to 18 carbon atoms in the alkyl portion and from 3 to 20, such as from 4 to 10, alkyl ether groups. Also usable are corresponding ethoxylation and/or propoxylation products of N-alkylamines, vicinal diols, fatty acid esters, and fatty acid amides which, with regard to the alkyl portion, correspond to the stated long-chain alcohol derivatives, and of alkyl phenols having from 5 to 12 carbon atoms in the alkyl radical.

Suitable anionic surfactants may include soaps and those containing sulfate or sulfonate groups having alkali ions as cations in a non-limiting embodiment. Usable soaps may include the alkali salts of saturated or unsaturated fatty acids having from 12 to 18 carbon atoms. Fatty acids of this kind may also be used in a not completely neutralized form. Usable sulfate-type surfactants include the salts of sulfuric acid semesters of fatty alcohols having from 12 to 18 carbon atoms and the sulfation products of the stated non-ionic surfactants having a low degree of ethoxylation. Usable sulfonate-type surfactants include linear alkylbenzenesulfonates having from 9 to 14 carbon atoms in the alkyl portion, alkanesulfonates having from 12 to 18 carbon atoms, and olefin sulfonates having from 12 to 18 carbon atoms, resulting from the reaction of corresponding monoolefins with sulfur trioxide, and alpha-sulfo fatty acid esters, resulting from the sulfonation of fatty acid methyl or ethyl esters.

Such surfactants may be present in the detergent composition to be applied to the textile substrate in amounts ranging from 5 wt. % to 50 wt. %, such as from 8 wt. % to 30 wt. % weight, based on the total weight of the detergent composition.

The detergent composition may also contain cationic surfactants. Cationic surfactants may include, but is not limited to, esterquats and/or quaternary ammonium compounds (QACs) according to general formula (R^{I})(R^{II})(R^{III})(R^{IV})N⁺X⁻, in which R^{I} to R^{IV}/represent C1-22 alkyl radicals, C7-28 arylalkyl radicals or heterocyclic radicals that are the same or different, where two radicals, or, in the case of aromatic bonding such as in pyridine, even three radicals form, together with the nitrogen atom, the heterocycle, for example a pyridinium or imidazolinium compound, and X⁻ represents halide ions, sulfate ions, hydroxide ions, or similar anions. Examples of suitable QACs are benzalkonium chloride (N-alkyl-N,N-dimethyl-benzylammonium chloride), benzalkone B (m, p-dichlorobenzyl-dimethyl-C12-alkylammonium chloride), benzoxonium chloride (benzyl-dodecyl-bis-(2-hydroxyethyl)-ammonium chloride), cetrimonium bromide (N-hexadecyl-N, N-trimethyl-ammonium bromide), benzetonium chloride (N,N-dimethyl-N-[2-[2-[p-(1,1,3,3-tetramethylbutyl)phenoxy]-ethoxy]ethyl]benzylammonium chloride), dialkyldimethylammonium chlorides such as di-n-decyl-dimethylammonium chloride, didecyldimethylammonium bromide, dioctyldimethylammonium chloride, 1-cetylpyridinium chloride and thiazoline iodide and mixtures thereof. Non-limiting examples of QACs are benzalkonium chlorides having C8-C18 alkyl radicals, such as C12-C14 alkylbenzyldimethyl ammonium chloride.

Suitable examples of ester quats are shown, for example, in formulas (I) and (II):

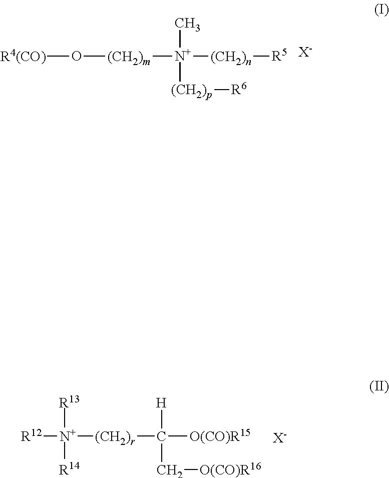

In formula (I) R⁴ is an aliphatic alk(en)yl radical having from 12 to 22 carbon atoms with 0, 1, 2 or 3 double bonds and/or optionally with substituents; R⁵ is H, OH or O(CO)R⁷, R⁶ is independently of R⁵H, OH or O(CO)R⁸, wherein R⁷ and R⁸ represent, independent of one another, an aliphatic alk(en)yl radical having 12 to 22 carbon atoms with 0, 1, 2 or 3 double bonds. m, n and p can each independently be 1, 2 or 3. X⁻ can be either a halide, methosulfate, methophosphate or phosphate ion, and mixtures of these anions. In a non-limiting example, R⁵ may represent the group O(CO)R⁷. In non-limiting embodiments, R⁵ is the group O(CO)R⁷ and R⁴ and R⁷ are alk(en)yl radicals having from 16 to 18 carbon atoms. In other non-limiting embodiments, R⁶ is OH. In formula (II), R¹², R¹³ and R¹⁴ represent, independent of one another, a C1-4 alkyl, alkenyl or hydroxyalkyl group, where R¹⁵ and R¹⁶ each may be independently selected as C8-28 alkyl group, X⁻ is an anion, and r is a number between 0 and 5. Examples of useful ester quats include methyl-N-(2-hydroxyethyl)-N, N-di(tallowacyloxyethyl)ammonium methosulfate, bis(palmitoyloxyethyl)hydroxyethyl methyl ammonium methosulfate, 1,2-bis[tallowacyloxy]-3 trimethylammoniumpropane chloride, methyl N,N-bis(stearoyloxyethyl)-N-(2-hydroxyethyl) ammonium methosulfate, or N,N-dimethyl-N, N-di(tallowacyloxyethyl)ammonium methosulfate.

However in non-limiting embodiments, the cationic surfactants, e.g. the ester quats, if present, may not present in the detergent composition but in a further composition.

Suitable bleaching agents for use in laundry articles include, such as, organic peracids or persalts of organic acids, such as phthalimidopercaproic acid, perbenzoic acid or salts of diperdodecanedioic acid, and inorganic salts releasing hydrogen peroxide under the washing conditions, including alkali perborate, alkali percarbonate, persilicate and/or persulfate, such as caroate. The peroxygen compounds may be used in the form of powders or granules, which may also be coated in a manner known in principle. The addition of small amounts of known bleaching agent stabilizers, such as phosphonates, borates or metaborates, metasilicates and magnesium salts, such as magnesium sulfate may be expedient. A bleach-containing detergent composition to be applied to the textile substrate contains in a non-limiting embodiment 15% by weight to 50% by weight, for example 18% by weight to 35% by weight, of peroxygen bleaching agent, such as alkali percarbonate.

In addition to the bleach, a bleach activator may be included, either as part of the bleach-containing detergent composition or as part of a further composition. As bleach activators it is possible, such as, to use compounds that give aromatic or aliphatic peroxycarboxylic acids under perhydrolysis conditions. Polyacylated alkylendiamines, such as tetraacetylethylenediamine (TAED); acylated triazine derivates, such as 1,5-diacetyl-2,4-dioxohexahydro-1,3,5-triazine (DADHT); acylated glycolurils, such as tetraacetylglycoluril (TAGU); N-acylimides, such as N-nonanoylsuccinimide (NOSI); acylated phenol sulfonates, such as n-nonanoyl or isononanoyl oxybenzene sulfonate (n- or iso-NOBS); carboxylic acid anhydrides, such as phthalic acid anhydride; and acylated polyhydric alcohols, such as triacetin; in non-limiting embodiments, the bleach activator may be ethylene glycol diacetate and 2,5-diacetoxy-2,5-dihydrofuran. It is also possible to use peritrile-forming nitriles, for example acetonitriles bearing ammonium groups, under perhydrolysis conditions.

In addition to or instead of conventional bleach activators, so-called bleach catalysts may be used. These substances are bleach-boosting transition metal salts or transition metal complexes such as, for example, Mn-, Fe-, Co-, Ru-, or Mo-salene complexes or -carbonyl complexes. Mn-, Fe-, Co-, Ru-, Mo-, Ti-, V-, and Cu-complexes with nitrogenous tripod ligands as well as Co-, Fe—Cu-, and Ru-amine complexes can also be used as bleach catalysts. Non-limiting bleach catalysts include complexes with triazacyclononane ligands, which may optionally be mono- or poly-alkyl, for example, 1,4,7-trimethyl-1,4,7-triazacyclononane.

The coated textile laundry article may be applied with microcapsules in a non-limiting embodiment, such as core-shell microcapsules containing an active ingredient. The microcapsules can be applied directly to the textile substrate by means of an adhesive or introduced by the detergent composition or further composition. In non-limiting embodiments, the active ingredient contained in the microcapsules may be a perfume, an enzyme, or a bleaching agent, such as a fragrance. In this case, all fragrances known to the person skilled in the art can be used. The microcapsule may be a melamine, acrylate, or polyvinyl alcohol-based microcapsule.

In various embodiments, the coated textile laundry article textile substrate comprises fibers which are provided with an active substance-containing water-soluble coating. In non-limiting embodiments, the water-soluble coating may be formed from a layer-forming material, such as but not limited to particulate sols and/or water-soluble polymers. Polyvinyl alcohols may be suitable as water-soluble polymers.

Due to the presence of a further composition which is solidified on the textile substrate, further functionalities can be added to the laundry article. Thus, in the case where the further solidified composition comprises a laundry care fabric, such as a fabric softening compound and/or a dryer-releasable fragrance, the further solidified composition may have a higher softening point than the solidified detergent composition and does not dissolve in the washing process. The laundry article may be transferred after the washing process together with the washed laundry in a dryer. In the dryer, the further composition may provide further laundry care effect.

In a non-limiting embodiment, the further composition is a textile and/or skin care composition.

In this context, a textile care composition is understood to mean any composition which gives textile fabrics treated therewith an advantageous effect, such as a textile softening effect, crease resistance, fragrance or the effect of reducing the harmful or negative effects which may result during cleaning and/or conditioning and/or wearing, such as fading, etc.

The textile care composition may be a textile softening composition and includes, for example, an ester quat, a fabric softening clay, a cationic polymer, or a mixture of these fabric softening compounds. Other suitable textile-care compounds include, for example, soil release polymers, anti-shrinkage agents, anti-crease agents, color transfer inhibitors, antimicrobial agents, germicides, fungicides, antioxidants, ironing aids, repellents, impregnating agents, and fragrances.

A skin care composition is understood to mean a composition having a skin care compound that upon contact of a textile with the washing agent, is absorbed onto the textile and, upon contact of the textile with the skin, is beneficial to the skin in comparison with a textile that has not been treated with the skin care composition. This benefit may include, for example, the transfer of the skin care compound from the textile to the skin, less water transfer from the skin to the textile, or less friction on the skin surface due to the textile. Suitable skin care compounds include, for example, waxes, plant extracts, higher fatty acids, higher fatty alcohols, esters, hydrocarbons, lipids, vitamins, sunscreens, or phospholipids.

The detergent composition and the further composition may in one embodiment be arranged at least partially overlapping on the textile substrate. In an alternative embodiment, the detergent composition and the further composition are arranged side by side on the textile substrate.

In a non-limiting embodiment, the detergent composition has a color different from the color of the further composition.

In non-limiting embodiments, the entire surface of the textile substrate need not be covered with the solidified detergent composition and optional further composition as long as the detergent composition is present in at least a portion of the substrate surface, of a zone, on the substrate.

The detergent composition and optional further compositions may be applied to one or both sides of the textile substrate. In non-limiting embodiments, the detergent composition and the optional further compositions are applied to one side of the textile substrate. This has the particular advantage that the consumer does not have to come into direct contact with the compositions when dosing the laundry article, but can easily handle the laundry article on the composition-free surface.

It is possible to apply the detergent composition as a liquid solution, slurry or paste to the textile substrate and then perform a drying step while removing the slurry or solvent, for example water, enough to solidify the detergent composition on the textile substrate by drying. However, the detergent composition may be applied as a heated melt on the textile substrate and then solidified by cooling in a non-limiting embodiment. Not all the detergent composition must be liquefied by melting; for example, it is possible for the melt to contain particulate solids such as inorganic builders.

### Example 1

Stock solutions of a polyorganosiloxane (DOW Corning AC-8066) in water were prepared (5, 10 and 20% by weight). These were each distributed on three beakers and woven fabric samples (viscose/lyocell (70/30) or viscose/polylactic acid (PLA) (70/30), each 5 cm×5 cm) were immersed in the solutions for 20 minutes. In total, six woven fabric samples were used for each stock solution (triple determination). The woven fabric samples were dried overnight on a filter sheet.

The dirt solution (FI dirt SBL-2004) for the subsequent washing tests was prepared according to the following procedure: 0.85 g of FI dirt were introduced into 6,000 g of water and stirred for about 1.5 hours at elevated temperature (stirring plate: 68° C.).

Beakers were provided on multiple stir plates and each was then filled with 200 mL of the dirt solution. The finished woven fabric samples as well as blanks without equipment were then added individually to a beaker. Thereafter, the stirring function was switched on and stirred constantly for 1 hour at room temperature. After taking out the samples, they were hung up to dry overnight.

The assessment was done visually by 3 persons by comparing the finished woven fabric sample with the corresponding blank.

TABLE 1
Dirt-trap tests with cloths treated with 5, 10 and 20% solution and
untreated cloths for comparison. The higher the value, the higher the
degree of graying of the fabric sample → Graying-inhibiting effect
on the laundry. 0 = untreated fabric sample before the washing test

| | 5% | Com- | 10% | Com- | 20% | Com- |
|---|---|---|---|---|---|---|
| | so- | pari- | so- | pari- | so- | pari- |
| | lution | son | lution | son | lution | son |
| Viscose/PLA | 2 | 1.6 | 1.6 | 1.4 | 2.3 | 1.6 |
| Viscose/Lyocell | 1.6 | 1 | 1.7 | 1.4 | | |

What is claimed is:

1. A coated textile laundry article comprising a water-insoluble textile substrate, wherein the water-insoluble textile substrate is exposed to at least one modified or unmodified polyorganosiloxane and/or at least one amorphous silicon dioxide.

2. The coated textile laundry article of claim 1, wherein the
at least one modified or unmodified polyorganosiloxane is selected from the group consisting of polyalkylsiloxanes, fluorosilicones, ether-modified silicones, polyether-modified silicones, and combinations thereof; or
at least one modified or unmodified polyorganosiloxane having a molecular weight ranging from 150 g/mol to 50,000 g/mol; or
at least one modified or unmodified polyorganosiloxane present in an amount ranging from 0.001 to 50% by weight, based on the total weight of the coated textile laundry article; or
combinations thereof.

3. The coated textile laundry article of claim 1, wherein the laundry article further comprises a detergent composition solidified on the textile substrate.

4. The coated textile laundry article of claim 1, wherein the textile substrate comprises fibers that have a drug-containing water-soluble coating.

5. The coated textile laundry article of claim 4, wherein the drug-containing water-soluble coating comprises a layer-forming material selected from the group consisting of particulate sols, water-soluble polymers, and combinations thereof.

6. The coated textile laundry article of claim 1, wherein the water-insoluble textile substrate is coated with microcapsules containing at least one active ingredient.

7. The coated textile laundry article of claim 6, wherein the microcapsules are attached to the textile substrate by an adhesive applied to the water-insoluble textile substrate.

8. The coated textile laundry article of claim 1, wherein the water-insoluble textile substrate comprises:
a woven or non-woven fabric;
cellulose, polyester, polyethylene, polypropylene, polyethylene terephthalate, polybutylene terephthalate, rayon, polyacrylic, polylactic acid, lyocell, viscose or nylon, or mixtures thereof; or
combinations thereof.

9. The coated textile laundry article of claim 1, wherein at least one detergent composition and at least one further composition are solidified on the water-insoluble substrate.

10. The coated textile laundry article of claim 9, wherein the at least one further composition is selected from the group consisting of textile compositions, skin care compositions, and combinations thereof.

11. The coated textile laundry article of claim 9, wherein the detergent composition and the further composition are arranged side by side on the water-insoluble textile substrate.

12. The coated textile laundry article of claim 9, wherein the detergent composition and the further composition are arranged at least partially overlapping each other on the water-insoluble textile substrate.

13. The coated textile laundry article of claim 1, wherein the coated textile laundry article is configured to be used for washing laundry, and wherein the graying of a laundry article is reduced in comparison to the same laundry article that is washing in the absence of the coated textile laundry article.

14. A method for machine or manual washing of laundry comprising:
adding a textile laundry article to a load of laundry; wherein the textile laundry article comprises a water-insoluble textile substrate, wherein the water-insoluble textile substrate is exposed to at least one modified or unmodified polyorganosiloxane and/or at least one amorphous silicon dioxide; and
washing the load of laundry.

15. A method for the preparation of a coated textile laundry article of claim 1, comprising:
providing the water-insoluble textile substrate,
subjecting the water-insoluble textile substrate to the at least one modified or unmodified polyorganosiloxane and/or the at least one amorphous silicon dioxide and optionally a detergent composition that is flowable when heated above 30° C. and optionally a further composition on at least one zone of the water-insoluble textile substrate; and
cooling and hardening the at least one modified or unmodified polyorganosiloxane and/or the at least one amorphous silicon dioxide and the optional detergent composition and the optional further composition on the water-insoluble textile substrate.
